(19) United States
(12) Patent Application Publication
(10) Pub. No.: US 2013/0316961 A1
(43) Pub. Date: Nov. 28, 2013

(54) TREATMENT OF MECP-2 ASSOCIATED DISORDERS

(71) Applicants: Jean-Christophe Roux, Allauch (FR); Laurent Villard, Marseille (FR); Frédéric Saudou, Orsay (FR)

(72) Inventors: Jean-Christophe Roux, Allauch (FR); Laurent Villard, Marseille (FR); Frédéric Saudou, Orsay (FR)

(73) Assignees: UNIVERSITÉ D'AIX-MARSEILLE, Marseille (FR); INSTITUT CURIE, Paris (FR); CENTRE NATIONAL DE LA RECHERCHE SCIENTIFIQUE, Paris (FR); INSERM (Institut National de la Santé et de la Recherche Médicale), Paris (FR)

(21) Appl. No.: 13/881,639

(22) PCT Filed: Oct. 24, 2011
(86) PCT No.: PCT/EP2011/068576

§ 371 Date: Aug. 5, 2013

(30) Foreign Application Priority Data
Oct. 25, 2010 (US) 61406248

(51) Int. Cl. C07C 323/25; C07D 491/06; A61K 38/13; A61K 45/06; C07K 7/64; A61K 31/145; A61K 31/436

(52) U.S. Cl. CPC C07C 323/25; A61K 31/145; C07D 491/06; A61K 31/436; A61K 45/06; C07K 7/645; A61K 38/13
USPC 514/20.5; 564/500; 514/665; 546/92; 514/291; 530/317

(57) ABSTRACT

The invention relates to the use of cystamine, cysteamine, or a salt thereof, or of calcineurin inhibitors for treating a MeCP2-associated disorder such as Rett syndrome.

### FIELD OF THE INVENTION

The present invention relates to compounds useful for treating neurodevelopmental and/or neurological disorders associated with MeCP2 expression defects.

### BACKGROUND OF THE INVENTION

The methyl-CpG-binding protein-2 (MeCP2) gene encodes a protein that is able to bind methylated DNA and to regulate the transcription of target genes. MeCP2 deficiency or overexpression causes severe neurodevelopmental and/or neurological diseases in humans.

They are several forms of neurodevelopmental and/or neurological disorders associated with MeCP2 expression defects. Rett syndrome, autism, pervasive development disorder, non-syndromic mental retardation, idiopathic neonatal encephalopathy and idiopathic cerebral palsy are typical examples of such disorders.

Rett syndrome is an X-linked dominant disorder, affecting females almost exclusively. Typically, affected girls may appear to develop normally until some point between 6 and 18 months of life, when developmental progress may cease or begin to regress. They lose purposeful hand use and any acquired language skills (both receptive and expressive), cranial growth slows, and repetitive hand movements develop. Other features such as ataxia, gait apraxia, seizures, breathing dysrhythmias (apnea or hyperpnea), and autistic behaviour may also emerge. They also suffer decreased somatic growth and wasting. Following this period of rapid deterioration, patients stabilize and then may recover some skills. Survival usually extends well into adulthood. Current estimates indicate that over 90% of cases of Rett syndrome are caused by a mutation in the MECP2 gene. Rett syndrome is diagnosed in approximately one in every 12,500 females alive at age 12 years.

Autism is a complex developmental disability that typically appears during the first three years of life. It is the result of a neurological disorder which affects the functioning of the brain. It has been found to be four times more prevalent in boys than girls. Typically, autistic children and adults have difficulties in verbal and non-verbal communication, social interactions, and leisure or play activities. Unable to learn from the natural environment as most children do, autistic child generally shows little interest in the world or people around him. Although some children with autism develop normally and even acquire advanced skills, most exhibit a wide range of behavioral problems. Autism affects the way a person comprehends, communicates and relates to others. Autism was originally thought to be primarily a psychiatric condition. However, further investigation showed that genetic and environmental factors are implicated in the pathogenesis of autism.

Pervasive developmental disorder (PDD) is an umbrella term characterizing neurodevelopmental disorders arised from abnormalities of brain development that can have a range of underlying genetic or environmental/biological causes, or arised from gene-environment interactions. PDD causes delays in the development of multiple basic functions, usually in child development, including socialization and communication. PDD may display as early as infancy and are usually evident by age three. Symptoms of PDD may include communication problems such as difficulty using and understanding language; difficulty relating to people, objects, and events; unusual play with toys and other objects; difficulty with changes in routine or familiar surroundings; and repetitive body movements or behavior patterns. Children with PDD vary widely in abilities, intelligence, and behaviors. Some children do not speak at all, others speak in limited phrases or conversations, and some have relatively normal language development.

Mental retardation is defined by an intellectual functioning level (IQ) below 70, significant limitations in two or more adaptive skill areas, and the condition present from childhood (defined as age 18 or less). Genetic etiologies are found in approximately two thirds of mental retardation cases. Biological processes involved in neuronal differentiation and synaptic plasticity, synaptic vesicle cycling and gene expression regulation are considered to be important in the causation of mental retardation. Most patients have the nonsyndromic form of the disorder, which is characterized by the absence of associated morphologic, radiologic, and metabolic features.

Idiopathic neonatal encephalopathy is an obstetric form of an encephalopathy from an unknown cause. Encephalopathy does not refer to a single disease, but rather to a syndrome of global brain dysfunction. The hallmark of encephalopathy is an altered mental state. Depending on the type and severity of encephalopathy, common neurological symptoms are loss of cognitive function, subtle personality changes, inability to concentrate, lethargy, and depressed consciousness. Other neurological signs may include myoclonus (involuntary twitching of a muscle or group of muscles), asterixis (abrupt loss of muscle tone, quickly restored), nystagmus (rapid, involuntary eye movement), tremor, seizures, jactitation (restless picking at things characteristic of severe infection), and respiratory abnormalities such as Cheyne-Stokes respiration (cyclic waxing and waning of tidal volume), apneustic respirations, and post-hypercapnic apnea.

Idiopathic cerebral palsy encompasses a group of non-progressive and non-contagious motor conditions, from an unknown cause, that cause physical disability in human development, chiefly in the various areas of body movement. Cerebral palsy is caused by damage to the motor control centers of the developing brain and can occur during pregnancy, during childbirth or after birth up to about age three. Resulting limits in movement and posture cause activity limitation and are often accompanied by disturbances of sensation, depth perception and other sight-based perceptual problems, communication ability, and sometimes even cognition; sometimes a form of cerebral palsy may be accompanied by epilepsy. Cerebral palsy, no matter what the type, is often accompanied by secondary musculoskeletal problems that arise as a result of the underlying etymology.

Of the many types of neurodevelopmental and/or neurological disorders associated with MeCP2, none of them has a known cure. Medications are used to address certain behavioral problems; therapy for children with neurodevelopmental and/or neurological disorder is usually specialized according to the child's specific needs.

MeCP2 is essential for normal brain development and an alteration of its expression causes severe cognitive, motor and autonomic dysfunction in humans. MeCP2 deficiency leads to a reduction in the number of axonal and dendritic processes and a decrease in dendritic spine density. (Armstrong, D. D. (2002) Neuropathology of Rett syndrome. Ment. Retard. Dev. Disabil. Res. Rev., 8, 72-76 and Belichenko, P. V., Wright, E. E., Belichenko, N. P., Masliah, E., Li, H. H., Mobley, W. C., Francke, U. (2009) Widespread changes in dendritic and axonal morphology in Mecp2-mutant mouse models of Rett syndrome: evidence for disruption of neuronal networks. J Comp. Neurol., 514, 240-258). Widespread abnormalities of dendrites and axons seem to be involved in MeCP2-associated disorders.

Several patents or applications disclose a variety of therapeutic compounds to treat MeCP2-associated disorders. For example, WO 2008/122087 describes agents that are able to ameliorate impaired microtubule dynamics, EP 1 559 447 describes the use of epothilones to treat autism by inducing tubulin polymerization into microtubules, U.S. Pat. No. 6,709,817 describes the use of trichostatin A, which is a histone deacetylase inhibitor, to compensate the vesicular transport by increasing tubulin acetylation and treating MeCP2-associated disorders. Patent application WO 2008/060375 describes the treatment of a mental retardation by augmenting BDNF level in brain.

Brain-derived neurotrophic factor (Bdnf) is one of the MeCP2 targets. Significant reductions in Bdnf mRNA and protein levels are found in MeCP2-deficient mice (Chang, Q., Khare, G., Dani, V., Nelson, S., Jaenisch, R. (2006) The disease progression of MeCP2 mutant mice is affected by the level of BDNF expression. Neuron. 49, 341-348). Bdnf acts on neurons in the central and peripheral nervous systems, supporting the survival of existing neurons and leading to the growth and differentiation of new neurons (Acheson, A., Conover, J. C., Fandl, J. P., DeChiara, T. M., Russell, M., Thadani, A., Squinto, S. P., Yancopoulos, G. D., Lindsay, R. M. (1995) A BDNF autocrine loop in adult sensory neurons prevents cell death. Nature., 6521, 450-4533). Furthermore, Bdnf plays a key role in axonal and dendritic differentiation and maturation throughout the entire brain (Huang, E. J., Reichardt, L. F. (2001). Neurotrophins: roles in neuronal development and function. Annu. Rev. Neurosci. 24, 677-736). Chalour and al, 2007 have shown that Bdnf is also involved in the regulation of cognitive, motor and autonomic functions which are all severely affected in patients suffering from neurological diseases caused by MeCP2 dysfunction, such as Rett syndrome (Chahrour, M. and Zoghbi, H. Y. (2007) The story of Rett syndrome: from clinic to neurobiology. Neuron. 56, 422-437 and Greenberg, M. E., Xu, B., Lu, B., Hempstead, B. L. (2009) New insights in the biology of BDNF synthesis and release: implications in CNS function. J. Neurosci. 29, 12764-12767).

Among the prior art, Sun et al, 2006 hypothesized that abnormal expression of Bdnf was a cause for the general neurological dysfunction occurring in the absence of MeCP2 (Sun, Y. E., Wu, H. (2006) The ups and downs of BDNF in Rett syndrome. Neuron. 49, 321-3). Chang et al, 2006 found that overexpression of Bdnf can improve the functional deficits observed in vivo in MeCP2-deficient mice (Chang, Q., Khare, G., Dani, V., Nelson, S., Jaenisch, R. (2006) The disease progression of MeCP2 mutant mice is affected by the level of BDNF expression. Neuron. 49, 341-348). These data suggest that Bdnf could play a key role in the appearance of the neurological phenotype caused by MeCP2 dysfunction. However, the link between the altered levels of MeCP2 and BDNF in the brain remained unclear.

### SUMMARY OF THE INVENTION

The purpose of the present invention is to provide new approaches for MeCP2-associated disorders treatment.

The present invention is founded on the identification that the MeCP2 protein plays a new role in the regulation of axonal BDNF and APP (Amyloid Protein Precursor) trafficking. More precisely, the inventors have shown that MeCP2 deficiency causes a loss of velocity of the BDNF and APP vesicles along the axons.

On the basis of these research findings, the inventors propose to modify the vesicular trafficking throughout the neurons using pharmacological molecules known as modulator of the vesicular trafficking, by acting as calcineurin inhibitors.

The invention provides compounds that increase the BDNF brain levels to provide more BDNF vesicules locally and to restore axonal BDNF trafficking.

According to a first aspect, the invention is directed to the use of cystamine or cysteamine, or a salt thereof, to treat a MeCP2-associated disorder.

In a particular embodiment, said cystamine or cysteamine is used to treat a MeCP2-associated disorder selected from Rett syndrome, autism, pervasive development disorder, non-syndromic mental retardation, idiopathic neonatal encephalopathy and idiopathic cerebral palsy.

Preferably, said MeCP2-associated disorder is Rett syndrome.

Said cystamine or cysteamine may be used alone or in combination with another pharmaceutically active compound.

According to a first aspect, the invention is directed to the use of a calcineurin inhibitor to treat a MeCP2-associated disorder in a human patient.

In a particular embodiment, the calcineurin inhibitor is a macrolide.

In a preferred embodiment, said calcineurin inhibitor is selected from the group consisting in calcipressins (also called regulators of calcineurin (ROAN) proteins), tacrolimus and tacrolimus analogs, cyclosporine A and cyclosporine A analogs, LxPV proteins, 2,6-diaryl-substituted pyrimidine derivatives and FK506-binding proteins.

In a more preferred embodiment, said calcineurin inhibitor is selected from the group consisting in calcipressin 1, calcipressin 2, calcipressin 3 (also called RCAN1, 2 and 3), tacrolimus (also called FK506 or Fujimycine), ascomycin, sirolimus, pimecrolimus, cyclosporine A, voclosporine (also called ISA247), LxPVc1, LxPVc2, LxPVc3, 6-(3,4-dichloro-phenyl)-4-(N,N-dimethylaminoethylthio)-2-phenyl-pyrimidine (also called CN585) and FK506-binding protein 8 (also called FKNP8).

In another preferred embodiment, said calcineurin inhibitor is tacrolimus.

In a preferred embodiment, said calcineurin inhibitor is cyclosporine A.

Calcineurin inhibitors of the invention are used to treat a MeCP2-associated disorder selected from Rett syndrome, autism, pervasive development disorder, non-syndromic mental retardation, idiopathic neonatal encephalopathy and idiopathic cerebral palsy.

In a preferred embodiment, said MeCP2-associated disorder is Rett syndrome.

Said calcineurin inhibitor may be used alone or in combination with another pharmaceutically active compound.

It is further described the use of a calcineurin inhibitor or cystamine, cysteamine, or a salt thereof, for the manufacture of a medicament for treating a MeCP2-associated disorder such as Rett syndrome. Methods of treatment are further disclosed, for treating a MeCP2-associated disorder, which methods comprise administering to the patient in need thereof, a therapeutically effective amount of a calcineurin inhibitor or cystamine, cysteamine, or a salt thereof.

### LEGEND OF THE FIGURES

FIG. 1. This graph represents the expression of several genes involved in Bdnf trafficking which are severely reduced in the Mecp2-deficient brain. Real-time quantitative PCR analysis of genes involved in Bdnf trafficking was performed on mRNA isolated from the rostral brain of P55 wild type (Wt, n=4 rostral brain; n=5 pons or medulla) and Mecp2-deficient mice (Ko, n=4 rostral brain; n=5 pons or medulla). Bdnf, Hap1, Htt, Sgk1, Dctn1, Dync1h1 and Ahi1 are all part of the machinery complex involved in the vesicular transport of Bdnf. Their transcripts are all importantly downregulated in the Mecp2-deficient brain. The expression of TrkB, the major Bdnf receptor, and Syt1, which is involved in vesicle fusion and recycling, is not affected by the absence of Mecp2. All values are expressed as relative mRNA levels normalized to Gapdh is the same sample and expressed as percentage of wild type level. * indicates statistically significant differences (p<0.05).

FIG. 2. This graph shows the postnatal expression of Hap1 and Htt throughout the brain of Mecp2-deficiency mice which is decreased. Quantification of the huntingtin-associated protein 1 (Hap1), and huntingtin (Htt) mRNAs in three areas of the mouse brain: rostral brain, pons and medulla oblongata. Quantifications were performed at P30 and P55 in wild type (Wt, n=4 at each age) and Mecp2-deficient mice (Ko, n=4 at each age). At P30, no deregulation was observed. At P55, Hap1 and Htt expression is strongly reduced in the three tested brain areas. All values are expressed as relative mRNA levels normalized to Gapdh is the same sample and expressed as percentage of wild type level. * Indicates statistically significant differences (p<0.05).

FIG. 3. This graph represents the repartition of the Bdnf protein between the striatum and the cortex of the Mecp2-deficient mouse which is abnormal. The Bdnf protein is present in the striatum and is anterogradelly transported from the cortex. We immunoquantified the staining levels in both areas (circles in A) and calculated a ratio between the striatum and the cortex (B). In the Mecp2-deficient brain, striatal Bdnf represents 45% of cortical Bdnf whereas it represents 75% in the wild type brain. This is suggestive of a defect of the axonal transport of Bdnf. * Indicates a statistically significant difference (p<0.05).

FIG. 4. This graph shows the anterograde and retrograde vesicular transport of Bdnf which is affected by Mecp2 level. Rat cortical neuronal cultures were electroporated with Bdnf-cherry, Mecp2-expressing vector or an empty vector and si-Mecp2 or si-control and plated on glass cover-sleep. After three days in vitro, Bdnf dynamics was analyzed by video-microscopy and anterograde and retrograde velocity was quantified and expressed in micrometers/minutes (mm/min) in the four conditions. * Indicates a statistically significant difference (p<0.05).

FIG. 5. This graph shows the App gene expression and the App fluorescence intensity which are not affected by Mecp2 level. The inventors dissected the forebrain of the Mecp2 deficient compared to Wild type mice. First they quantified the level of App mRNA by real time PCR and they did not found any significant changing and second they performed immunostaining in the same area, and also, did not found any significant changing.

FIG. 6. This graph shows the anterograde and retrograde vesicular transport of App which is affected by Mecp2 level. Rat cortical neuronal cultures were electroporated with Bdnf-cherry, and in addition an empty vector and si-Mecp2 or si-control and plated on glass cover-sleep. After three days in vitro, App dynamics was analyzed by video-microscopy and anterograde and retrograde velocity was quantified and expressed in micrometers/minutes (mm/min) in the four conditions. * Indicates a statistically significant difference (p<0.05).

FIG. 7A. This graph shows that chronic treatment with cysteamine extended the lifespan of Mecp2-deficient mice. The survival of Mecp2-deficient mice was assessed in animals treated with the water (vehicle, KO VEH) or cysteamine (KO CYST). The inventors found that cysteamine oral treatment significantly lengthened the life span of Mecp2-deficient mice (vehicle group: 65±2.2 days; cysteamine group: 74.8±5.2 days; p<0.05, Kaplan-Meir log-rank test). n=42 Vehicle-treated and n=17 cysteamine-treated Mecp2-deficient mice.

FIGS. 7B. and 7C. These graphs show the impact of the oral cysteamine treatment on the motor performances of Mecp2-deficient mice. The graphs show the behavioral performances in the vehicle group and cysteamine treated animals (n=10 cysteamine, KO CYST, n=10 vehicle, KO VEH). FIG. 7B: The total distance traveled by the mice in the open field arena during the 15-minute session is statistically increased in Mecp2-deficient mice treated with cysteamine from P75. FIG. 7C: The mouse velocity of Mecp2-deficient mice treated with cysteamine is significantly increased from P65 (*p<0.05).

### DETAILED DESCRIPTION OF THE INVENTION

The inventors have shown that a number of transcripts involved in Bdnf trafficking were consistently deregulated in MeCP2 deficiency animals. Furthermore, the inventors have found substantially reduced levels of the proteins of two key contributors to Bdnf trafficking (huntingtin and huntingtin-associated protein 1) in MeCP2 deficiency animals. Additionally, they observed that the velocity of Bdnf-containing vesicles in MeCP2-deficient axons was altered and that the phenotype could be rescued by MeCP2 expression.

These data indicate that restoring the BDNF trafficking is useful in treating MeCP2-associated disorders. The inventors now propose two ways to restore BDNF trafficking: First, by using cystamine and/or cysteamine to increase BDNF levels via HSJ1b and transglutaminase, second, by using a calcineurin inhibitor to modify the vesicular trafficking throughout the neurons.

According to the invention, a treatment of MeCP2-associated disorders can comprise the administration of therapeutically effective amount of cystamine and/or cysteamine. and/or a therapeutically effective amount of a calcineurin inhibitor

Neurodevelopmental and/or Neurological Disorders

Within the context of the invention, “subject” or “patient” means a mammal, particularly a human, wathever its age or sex, suffering of a neurodevelopmental and/or neurological disorder associated with MeCP2. The term specifically includes domestic and common laboratory mammals, such as non-human primates, felines, canines, equines, porcines, bovines, goats, sheep, rabbits, rats and mice. Preferably the patient to treat is a human being, including a child or an adolescent.

Within the context of this invention, “MeCP2-associated disorder” refers to a neurological and/or neurodevelopmental disorder that is caused by MeCP2 expression defects, abnormalities or anomalies and/or MeCP2 gene mutation defects, abnormalities or anomalies. Examples of such disorders include, without limitation, Rett syndrome, autism, pervasive development disorder, non-syndromic mental retardation, idiopathic neonatal encephalopathy and idiopathic cerebral palsy.

### Cystamine and Cysteamine Compounds

Within the context of this invention, “cystamine” refers to an organic disulfide with the following formula

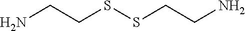

Any pharmaceutically acceptable salt thereof may be used, in particular the dihydrochloride salt.

Within the context of this invention, “cysteamine” refers to the chemical compound with the following formula

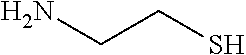

Any pharmaceutically acceptable salt thereof may be used, the hydrochloride salt.

### Calcineurin Inhibitor

Within the context of this invention, “calcineurin inhibitors” refer to compounds having a calcineurin inhibitory activity. Calcineurin is a calcium/calmodulin-regulated protein phosphatase involved in intracellular signaling. Preferably, the calcineurin inhibitors of the invention are macrolides. Calcineurin inhibitors of the invention can be selected on the group consisting in calcipressins (also called regulators of calcineurin (ROAN) proteins), tacrolimus and tacrolimus analogs, cyclosporine A and cydosporine A analogs, LxPV proteins, 2,6-diaryl-substitued pyrimidine derivatives and FK506-binding proteins. For example, calcineurin inhibitors of the invention can be calcipressin 1, calcipressin 2, calcipressin 3 (also called RCAN1, 2 and 3), tacrolimus (also called FK506 or Fujimycine), ascomycin, sirolimus, pimecrolimus, cyclosporine A, voclosporine (also called ISA247), LxPVc1, LxPVc2, LxPVc3, 6-(3,4-dichloro-phenyl)-4-(N,N-dimethylaminoethylthio)-2-phenyl-pyrimidine (also called CN585) and FK506-binding protein 8 (also called FKNP8).

Within the context of this invention, “macrolide” refers to a group of drugs whose activity stems from the presence of a macrolide ring. A macrolide ring is a large macrocyclic lactone ring to which one or more deoxy sugars, usually cladinose and desosamine, may be attached. The lactone rings are usually 14-, 15-, or 16-membered.

### Dosage

Within the context of the invention, a “therapeutically effective amount” means a dosage sufficient to produce a desired result. Generally, the desired result is an increasing and/or a restoration of BDNF or APP trafficking and/or availability. A therapeutically effective amount of an agent is not necessarily required to cure a disorder but will ameliorate a disorder or a treatment to prevent the onset of a disorder. Naturally, the amount which constitutes a “therapeutically effective amount” will vary depending on the compound, the disorder and its severity, and the age of the patient to be treated, that can be determined routinely by one ordinary skill in the art having regard to his own knowledge and to this disclosure.

For example, the therapeutically effective amount in calcineurin inhibitor or in cystamine or in cysteamine may be from about 0.01 mg/Kg/dose to about 100 mg/Kg/dose. Preferably, the therapeutically effective amount may be from about 0.01 mg/Kg/dose to about 25 mg/Kg/dose. More preferably, the therapeutically effective amount may be from about 0.01 mg/Kg/dose to about 10 mg/Kg/dose. Most preferably, the therapeutically effective amount may be from about 0.01 mg/Kg/dose to about 5 mg/Kg/dose. Therefore, the therapeutically effective amount of the active ingredient contained per dosage unit (e.g., tablet, capsule, powder, injection, suppository, teaspoonful and the like) as described herein may be from about 1 mg/day to about 7000 mg/day for a subject, for example, having an average weight of 70 Kg.

Within the context of the invention, “to increase BDNF trafficking” or “to restore BDNF trafficking” refers to any method/process that increases the trafficking and/or availability of BDNF in the brain. This includes, but is not limited to, the administration of modulator of the vesicular trafficking acting as a calcineurin inhibitor and/or the administration of cystamine and/or cysteamine.

### Pharmaceutical Composition

For the use according to the invention, the calcineurin inhibitor, cystamine and cysteamine can be formulated by methods known in the art. Compositions for the oral, rectal, parenteral or local application can be prepared in the form of tablets, capsules, granulates, suppositories, implantages, sterile injectable aqueous or oily solutions, suspensions or emulsions, aerosols, salves, creams, or gels, retard preparations or retard implantates. The calcineurin inhibitor, cystamine and cysteamine may also be administered by implantable dosing systems or by perfusion.

### Route of Administration

A calcineurin inhibitor, cystamine and cysteamine according to the invention or a pharmaceutical composition thereof may be administered by any conventional route of administration including, but not limited to oral, pulmonary, intraperitoneal (ip), intravenous (iv), intramuscular (i1), subcutaneous (sc), transdermal, buccal, nasal, sublingual, ocular, rectal and vaginal. In addition, administration directly to the nervous system may include, and are not limited to, intracerebral, intraventricular, intracerebroventricular, intrathecal, intracistemal, intraspinal or peri-spinal routes of administration by delivery via intracranial or intravertebral needles or catheters with or without pump devices. It will be readily apparent to those skilled in the art that any dose or frequency of administration that provides the therapeutic effect described herein is suitable for use in the present invention.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate.

The below examples illustrate the invention without limiting its scope.

### EXAMPLES

### Materials and Methods

### Animals

Experiments were performed on Sprague dawley rats and B6.129P2(C)-MeCP2 μm1-1Bird mouse model for RTT (Guy, J., Hendrich, B., Holmes, M., Martin, J. E., Bird, A. (2001) A mouse Mecp2-null mutation causes neurological symptoms that mimic Rett syndrome. Nat. Genet. 27, 322-326). The mice were obtained from the Jackson Laboratory and maintained on a C57BL/6 background. The rats were obtained from Charles River Laboratories, France. The experimental procedures were carried out in keeping with the European guidelines for the care and use of laboratory animals (Council Directive 86/6009/EEC). For the mouse experiments, hemizygous mutant males (MeCP2^{-/y}) were generated by crossing heterozygous knockout females to C57BL/6 males. Genotyping was performed by routine PCR technique according to Jackson Laboratory protocols and as previously described (Viemari, J. C., Roux, J. C., Tryba, A. K., Saywell, V., Burnet, H., Peña, F., Zanella, S., Bévengut, M., Barthelemy-Requin, M., Herzing, L. B., Moncla, A., Mancini, J., Ramirez, J. M., Villard, L., Hilaire, G. (2005) Mecp2 deficiency disrupts norepinephrine and respiratory systems in mice. J. Neurosci. 25, 11521-11530).

Cell Culture (Transfection siRNA, Expression Vector) and Videomicroscopy

Cortices of E17 rat embryos were dissected and dissociated as previously described (Saudou, F., Finkbeiner, S., Devys, D., Greenberg, M. E. (1998) Huntingtin acts in the nucleus to induce apoptosis but death does not correlate with the formation of intranuclear inclusions. Cell. 95, 55-66). Cortical neurons were electroporated with the rat neuron Nucleofector® kit according to the supplier's manual (Amaxa, Biosystem, Köln, Germany), and plated on poly-I-lysin coated cover-sleeps (Sigma). Plasmids used: pcDNA3 empty vector (Invitrogen), Bdnf-mCherry, a generous gift from G. Banker (Oregon Health and Science University, Portland, Oreg.), MeCP2 expression vector, a generous gift from N. Landsberger (University of Busto Arsizio), GFP (Amaxa, Biosystem, Köln, Germany). SiRNA used: rat MeCP2 (Sigma), scramble RNA control (Eurogentec). Neuronal cultures were kept in Neurobasal medium supplemented with B27 and Glutamax (GIBCO). After three days in vitro, neurons were used for Bdnf motility assay and/or for immunofluorescence staining. For motility assays, live videomicroscopy was carried out using an imaging system previously detailed (Gauthier, L. R., Charrin, B. C., Borrell-Pagès, M., Dompierre, J. P., Rangone, H., Cordelières, F. P., De Mey, J., MacDonald, M. E., Lessmann, V., Humbert, S., Saudou, F. (2004) Huntingtin controls neurotrophic support and survival of neurons by enhancing BDNF vesicular transport along microtubules. Cell. 118, 127-138). Cells were grown on a glass coverslip that was mounted in a Ludin chamber. The microscope and the chamber were kept at 37° C. Stacks of 5-7 images with a Z-step of 0.3 μm were acquired with a 100× PlanApo N. A. 1.4 oil immersion objective coupled to a piezo device (PI). Images were collected in stream mode using a Micromax camera (Ropper Scientific) set at 2×2 binning with an exposure time of 50 to 150 ms (frequency of 1 stacks/s). All stacks were treated by automatic batch deconvolution using the PSF of the optical system. Projections, animations and analyses were generated using ImageJ software (http://rsb.info.nih.gov/ij/, NIH, USA). Dynamics were characterized by tracking positions of GFP vesicles in cells as a function of time with a special plug-in (F. P. Cordelieres, IC, http://rsb.info.nih.gov/ij/plugins/track/track.html). During tracking, the Cartesian coordinates of the centers of vesicles were used to calculate dynamic parameters (velocity, directionality).

For immunostaining, cell were fixed with PFA 4% and processed with standard protocol. The antibodies used: MeCP2 (upstate), tubulin clone 6-11B-1 (Sigma), acetyltubulin (Sigma). Anti-mouse and anti-rabbit secondary antibodies conjugated to AlexaFluor-488 and AlexaFluor-555 were purchased from Molecular Probes (Invitrogen). Images were acquired with Leica SP5 confocal microscopy and the staining intensity was analyzed with ImageJ software by measuring the mean nuclear intensity (http://rsb.info.nih.gov/ij/, NIH, USA).

### Microarray Protocol and Analysis

P55 wild type (n=3) and MeCP2-deficient (n=3) mice were sacrificed by cervical dislocation. The medulla oblongata were dissected and immediately placed at −80° C. Frozen medulla were placed in 2 ml tubes in liquid nitrogen and tissues were homogenized by pulverization using a potter. Total RNA was extracted using TRIzol reagent (Invitrogen) according to the manufacturer's instructions. RNA samples were treated with 0.5 units of DNase I, RNase free (Qiagen) for 1 μg RNA at 37° C. during 30 min followed by an enzyme inactivation at 65° C. during 5 min. The quality and purity of the RNA was analyzed on an Agilent 2100 Bioanalyzer (Agilent Technologies, Palo Alto, Calif.) according to the manufacturer's instructions and using the Agilent 2100 Bioanalyzer Software.

First strand cDNA was prepared from 500 ng of total RNA from each pool of wild type and MeCP2-deficient brainstems in the presence of Cy3 or Cy5 dCTP respectively, and hybridized to the mouse 60-mer oligo microarray (Agilent Mouse Development Oligo Microarray 2×22K). Hybridization compared the Cy5-CTP-labeled MeCP2 deficient target to the Cy3-CTP-labeled wild type target and was carried out using the Agilent Gene Expression Hybridization Kit (G4121A), following the manufacturer's instructions. Briefly, 500 ng of treated sample cRNA was mixed with 500 ng of control cRNA and this solution was subjected to fragmentation (30 min at 60° C.) and then hybridized to the arrays in a rotary oven (65° C., 17 h, 10 RPM). Slides were disassembled and washed in solutions I and II according to the manufacturer's wash buffer kits instructions, and scanned with the Agilent DNA Microarray Scanner model G2505B (Agilent Technologies). Normalization was performed using a LOWESS algorithm, and dye-normalized signals were used to calculate log ratios. Features with reference values of < or >2 SDs above background for the negative control features were regarded as missing values. We only considered genes that were constantly deregulated in two independent experiments (n=3 Ko and n=3 Wt for each experiment), using the dye swap for each protocol. Analysis of variance (ANOVA) and replicate averaging was performed as previously described (Carter, M. G., Hamatani, T., Sharov, A. A., Carmack, C. E., Qian, Y., Aiba, K., Ko, N. T., Dudekula, D. B., Brzoska, P. M., Hwang, S. S., Ko, M. S. (2003) In situ-synthesized novel microarray optimized for mouse stem cell and early developmental expression profiling. Genome. Res. 13, 1011-1021) and for significance analysis of microarrays a t-test algorithm and a Benjamin-Hochberg correction was applied on raw data.

### RNA Extraction and RNA Quantification

Total RNA was extracted from the medulla oblongata, the pons or the midbrain-hindbrain samples coming from P55 and P30 old mice. Samples were collected in a 2 ml tube in liquid nitrogen and tissues were homogenized by pulverization using a potter. Total RNA was extracted using TRIzol reagent (Invitrogen) according to the manufacturer's instructions. RNA samples were treated with 0.5 Kunitz of Dnase I, Rnase free (Qiagen) for 1 μg RNA at 37° C. during 30 min followed by an enzyme inactivation at 65° C. during 5 min. The quality and purity of the pontine RNA samples were traited with 1 unit of Dnase I (Roche) for 1 μg RNA 37° C. during 30 min followed by an enzyme inactivation of 5 min at 75° C. Reverse transcription of 500 ng of total RNA was performed in 22 μl of Superscript reaction buffer containing 12.5 ng de dN6, 40 U of Rnase inihibitor (Promega), 10 mM dNTP and 200 U of Superscript II reverse transcriptase (Invitrogen). For quantitative PCR reaction we used the LightCycler 480 system (Roche) and we choosen the SYBR Greenl Master kit (Roche). Each reaction was performed in triplicate for 2 μl cDNA (½ dilution for the first-strand reaction) and 200 nM of each primer. First, we decided to confirm if the 12 genes identified as deregulated in the microarray protocol were deregulated using real time PCR. We also quantified mRNA expression of the main genes involved in the axonal transport of the Bdnf using a pair of specific primers transcript. As an internal control, Gapdh was measured under the same conditions in real-time quantitative PCR using a pair of primers specific for Gapdh. The Ct value is the threshold cycle at which signal become upper than the background noise in function of a threshold determined during the exponential phase of amplification. To normalize our results and quantify expression of different genes of interest the inventors have used the deltaCt (ΔCt) method in comparing the Ct between interest gene and internal control. ΔCt=Ct gene of interest−Ct Gapdh.

### Immunostaining and Immuno Quantification

Adult (P55) male were anesthetized with a lethal pentobarbitone injection (100 mg kg-1 i.p.) and transcardially perfused (chilled saline for 1 min followed by PBS 0.1M containing 4% paraformaldehyde for 10 min). Brains were postfixed for 5 h and placed overnight in PBS containing 20% sucrose and frozen at −80° C. Coronal sections (20 μm) of the brain were cut using a cryostat (Microm, France) and one every successive five slices was arranged serially on a slide. The sections were permeabilized (0.15% TritonX-100), blocked with 7% normal serum (rabbit), and incubated overnight at 4° C. with the primary antibody in PBS containing 3.5% serum, 0.15% Triton X-100. The sections were washed, incubated with the secondary antibody in PBS containing 3.5% normal serum, 0.15% Triton X-100, and re-washed. The slides were subsequently mounted in prolong antifade (Thermo Electron). Hap1 was probed with respectively affinity-purified mouse monoclonal antibody (1:500, BD bioscience H89720). Htt was probed with affinity-purified mouse antibody (1:1000, Euromedex HU-4C8-As). Goat-anti-rabbit alexa 546 (1:400) (Molecular Probes, Eugene Oreg.) was used as secondary antibody. The immunolabeled slides were digitized and recorded using a Leica DMR microscope (Leica Microsystems, Wetzlar, Germany) equipped with a CooISNAP camera (Princeton, Trenton, N.J., USA). To immunoquantify Htt or Hap1 proteins, even if this method does not provide an absolute value, we were very cautious to compare the intensity of staining between wild type and MeCP2^{-/y} tissues. We dissected the different tissues the same day and all the following steps, like fixation, freezing, cutting, staining and finally scanning the pictures were done with the same conditions, solutions, timing by alternating tissues coming from both groups. To avoid any changing of light intensity, the first slice scanned at the beginning of a single day was scanned again at the end. The linearity of the camera response was checked and we carefully selected the range between the lowest and the highest levels of fluorescence intensity in order to never reach saturation. All pictures were converted in grayscale. Densitometric analysis (luminance) of the staining was performed applying ImageJ software from the National Institutes of Health (http://rsb.info.nih.gov). The Optical density was calculated as OD=log 10 (255/luminance).

### Protein Isolation and Western Blot Analysis.

Tissues were extracted by sonication and then proteins were isolated in a lysis buffer containing 20 mM Tris-HCL (ph=7.5), 150 mM NaCI, 2 mM EGTA, 0.1% Triton X-100 and complete protease inhibitor tablet (Roche). Proteins concentrations were determined by the BCA (Bicinchoninic acid) method. After a denaturating step at 96° C. for 5 min, proteins (20 μg) were separated on a 8% SDS-polyacrylamide gel and transferred onto a PVDF membrane (Amersham Pharmacia Biotech) by liquid electroblotting (Bio-Rad) for 1 h at 100V. Non specific binding was achieved by preincubating the membrane with 5% nonfat dry milk in TBS Tween 0.1% for 1 h at room temperature. Primary antibodies for Hap1 (1/5000 mouse, BD Bioscience H89720), Htt antibody (1/5000, mouse Euromedex HU-4C8-As) or GAPDH (1/5000, Millipore), over night 4° C. were diluted in the same solution and incubated overnight at 4° C. After extensive washing of the membrane with 5% nonfat dry milk TBS Tween 0.1%, appropriate peroxydase-conjugate antibody were incubated 2 h at room temperature. Bound peroxydase-conjugate antibody was revealed with the enhanced chemiluminescence reagent kit (Supersignal West Femto, Pierce). Films were digitized by a camera, and signal quantified on 16-bits images using the ImageJ software from NIH.

### In Vivo Cysteamine Treatment of Mecp2-Deficient Mice

From P30, animals received a cysteamine treatment in their drinking water (225 mg/kg/day) (Sigma-Aldrich; St-Quentin, France). Every 2 days, mice were weighted, cysteamine solution renewed and treatment consumption checked. Adding cysteamine to drinking water did not significantly affect water consumption (4.1±0.2 and 4.2±0.1 ml/day for untreated and treated Mecp2-deficient mice). Seventy-nine Mecp2 deficient mice were studied (lifespan study, n=17 cysteamine-treated and n=42 untreated; motor evaluation n=10 cysteamine-treated and n=10 untreated). The mice were assigned to each experimental group by an investigator who was blind to their genotype. Open-field activity was measured in an arena made of clear perspex (38×30 cm). The test session lasted 15 min with data recorded using the Ethovision 2.3.19 tracking system (Noldus Information Technology). Velocity (cm/s) and total distance moved (cm) were recorded. Velocity calculations on Ethovision were obtained using an input filter setting the minimal distance moved (0.6 cm) so that ambulations shorter than this value were never taken into account.

### Satistical Analysis

Statview software (SAS Institute Inc., Cary, N. C., USA) was used for statistical analysis. Data are expressed as mean±SEM.

### Example 1

### Transcriptional Profiling in the Medulla Oblongata of MeCP2-Deficient Mice Showed Abnormal Expression of a Number of Genes Involved in Bdnf Trafficking

The inventors have analysed mRNA expression patterns in symptomatic P55 MeCP2-deficient (Ko) (n=6) and wild type mice (Wt) (n=6) medulla oblongata using DNA microarrays. Each sample was hybridized independently and using dye swap. They have found 12 genes to be deregulated in all MeCP2-deficient samples (table 1). To confirm the microarray findings, they have used new MeCP2-deficient medulla oblongata samples (n=3) to measure transcript levels using real-time quantitative PCR. The expression of 10 of the 12 genes was confirmed to be deregulated in the medulla oblongata of all MeCP2-deficient mice tested. These ten genes included two known to play an active role in neuronal transport of Bdnf by huntingtin-associated protein 1 (Hap1) and serum/glucocorticoid-regulated kinase 1 (Sgk1). Real-time PCR experiments found a 35% reduction in Hap1 mRNA and a 55% reduction in Sgk1 mRNA. Hap1 can form a complex with huntingtin protein (Htt) and this interaction is directly involved in Bdnf trafficking (Gauthier, L. R., Charrin, B. C., Borrell-Pagès, M., Dompierre, J. P., Rangone, H., Cordelières, F. P., De Mey, J., MacDonald, M. E., Lessmann, V., Humbert, S., Saudou, F. (2004) Huntingtin controls neurotrophic support and survival of neurons by enhancing BDNF vesicular transport along microtubules. Cell. 118, 127-138). Sgk1 is known to phosphorylate Htt and modulate Htt function (Rangone, H., Poizat, G., Troncoso, J., Ross, C. A., MacDonald, M. E., Saudou, F., Humbert, S. (2004) The serum- and glucocorticoid-induced kinase SGK inhibits mutant huntingtin-induced toxicity by phosphorylating serine 421 of huntingtin. Eur. J. Neurosci. 19, 273-279). The inventors have also found deregulation of the type 1 inositol 1,4,5-triphosphate receptor (Itpr1), a gene involved in the regulation of the general axonal trafficking process via physical interaction with a complex containing Hap1 and Htt proteins (Tang, T. S., Tu, H., Chan, E. Y., Maximov, A., Wang, Z., Wellington, C. L., Hayden, M. R., Bezprozvanny, I. (2003) Huntingtin and huntingtin-associated protein 1 influence neuronal calcium signaling mediated by inositol-(1,4,5) triphosphate receptor type 1. Neuron. 39, 227-239). While expression of both Hap1 and Sgk1 is reduced in the medulla oblongata of MeCP2-deficient mice, Itpr1 mRNA levels are more than doubled (+109%).

TABLE 1
Deregulated genes in the medulla oblongata of MeCP2 deficient mice

| Symbol | Name | Accession | Expression |
|---|---|---|---|
| Cdkn1a | Cyclin-dependent kinase inhibitor 1A | NM_007669 | 33% |
| Ddit4 | DNA-damage-inducible transcript 4 | NM_029083 | 56% |
| Fxyd6 | FXYD domain containing ion transport regulator 6 | NM_022004 | 71% |
| Hap1 | Huntingtin-associated protein 1 | NM_010404 | 72% |
| Mt2 | Metallothionein 2 | NM_008630 | 65% |
| Nfkbia | Nuclear factor of kappa light chain gene enhancer in B- | NM_010907 | 51% |
| | cells inhibitor, alpha | | |
| Sgk1 | Serum/glucocorticoid regulated kinase 1 | NM_011361 | 56% |
| Atp5a1 | ATP synthase, H+ transporting, mitochondrial F1 | NM_007505 | 158% |
| | complex, alpha subunit, isoform 1 | | |
| Cnot7 | CCR4-NOT transcription complex, subunit 7 | NM_011135 | 154% |
| Itpr1 | Inositol 1,4,5-triphosphate receptor 1 | NM_010585 | 139% |
| Pacsin2 | Protein kinase C and casein kinase substrate in | NM_011862 | 133% |
| | neurons 2 | | |
| Spp1 | Secreted phosphoprotein 1 | NM_009263 | 144% |

### Example 2

### Underexpression of Genes Involved in Bdnf Trafficking in the Whole Brain of MeCP2-Deficient Mice

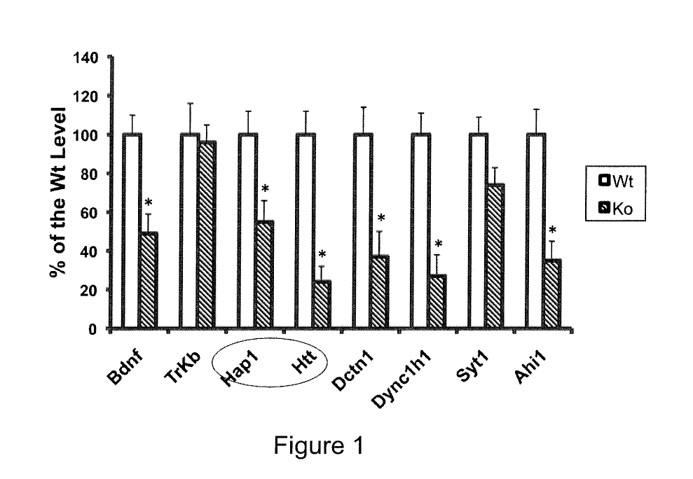

To determine if the findings made analyzing the medulla oblongata could be extended to other regions of the MeCP2-deficient brain, the inventors have tested new samples to see whether Hap1, Sgk1 and Itpr1 transcripts were abnormally expressed in different regions of the brain at P55. Using real-time quantitative PCR on dissected brain areas, they have found Hap1 to be significantly downregulated in the medulla oblongata (−47%, n=5) and also in the pons (−42%, n=4) and the rostral brain (−45%, n=4) of MeCP2-deficient mice (FIG. 1). The inventors have confirmed that Sgk1 was downregulated in the medulla oblongata (−50%, n=5) and observed a downregulation in the pons (−37%, n=5). The have found a significant upregulation in the rostral brain (+44%, n=4). They have also confirmed that Itpr1 was upregulated in the medulla oblongata (+110%, n=5), the pons (+150%, n=5) and the rostral brain (+220%, n=4). They have measured Bdnf mRNA levels in the brain of MeCP2-deficient mice. They have found Bdnf to be underexpressed in the medulla oblongata (−48%, n=5) and also in the pons (−53%, n=5) and the rostral brain (−51%, n=4) of MeCP2-deficient animals. The inventors have extended the expression analysis to other genes known to be involved in the neuronal trafficking of Bdnf: huntingtin (Htt), dynactin 1 (Dctn1), dynein cytoplasmic 1 heavy chain 1 (Dync1h1) and abelson helper integration site 1 (Ahi1). For this analysis, they have used samples from rostral brain regions as they are known to contain the highest levels of these genes (Chan, E. Y., Nasir, J., Gutekunst, C. A., Coleman, S., Maclean, A., Maas, A., Metzler, M., Gertsenstein, M., Ross, C. A., Nagy, A., Hayden, M. R. (2002) Targeted disruption of Huntingtin-associated protein-1 (Hap1) results in postnatal death due to depressed feeding behavior. Hum Mol Genet. 11, 945-959 and Dragatsis, I., Zeitlin, S., Dietrich, P. (2004) Huntingtin-associated protein 1 (Hap1) mutant mice bypassing the early postnatal lethality are neuroanatomically normal and fertile but display growth retardation. Hum Mol Genet. 13, 3115-3125). They have found that mRNA levels of Htt (−76%, n=4), Dctn1 (−63%, n=4), Dync1h1 (−73%, n=4) and Ahi1 (−65%, n=4) were greatly reduced in the MeCP2-deficient brain.

### Example 3

### Absence of MeCP2 Causes Gradual Postnatal Underexpression of Htt and Hap1 Throughout the Brain

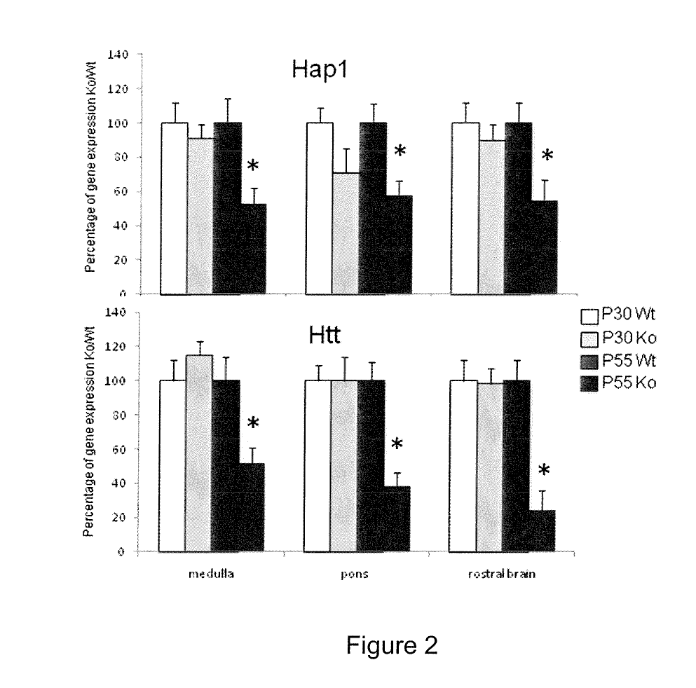

MeCP2-deficient mice are normal until one month of age when they start to gradually develop motor and cognitive dysfunction (Guy, J., Hendrich, B., Holmes, M., Martin, J. E., Bird, A. (2001) A mouse Mecp2-null mutation causes neurological symptoms that mimic Rett syndrome. Nat. Genet. 27, 322-326). The inventors have hypothesized that the progressive neurological dysfunction observed in MeCP2-deficient mice might be caused by progressive postnatal alteration of Hap1 and Htt expression, which, in turn, induces progressive alteration of Bdnf trafficking and the associated neurological abnormalities. They have used new MeCP2-deficient brain samples (n=4 in both experimental and control groups) collected at P30, before the appearance of the first phenotypic manifestations. Using real-time quantitative PCR, they have measured Hap1 and Htt mRNA levels in the medulla oblongata, pons, and rostral brain and have found that Hap1 and Htt expression was not affected at P30 in MeCP2-deficient animals (FIG. 2). The same experiments performed at P55 (n=4) found a marked decrease in the expression of Htt and Hap1 in all three areas, indicating postnatal progression of the phenotypic manifestations (FIG. 2).

### Example 4

### Decreased Levels of Htt and Hap1 Protein in the MeCP2-Deficient Brain

To determine whether the abnormal mRNA levels measured in the MeCP2-deficient brain were associated with abnormal protein levels, the inventors have used immunohistofluorescence to quantify Htt and Hap1 proteins in new brain samples (n=4) and the staining showed very low levels of both proteins in different parts of the MeCP2-deficient brain. Immunoquantification was performed in several brain areas carefully selected for the reason that both proteins are particularly abundant (Fujinaga, R., Kawano, J., Matsuzaki, Y., Kamei, K., Yanai, A., Sheng, Z., Tanaka, M., Nakahama, K., Nagano, M., Shinoda, K. (2004) Neuroanatomical distribution of Huntingtin-associated protein 1-mRNA in the male mouse brain. J. Comp. Neurol. 478, 88-109 and Kotliarova, S., Jana, N. R., Sakamoto, N., Kurosawa, M., Miyazaki, H., Nekooki, M., Doi, H., Machida, Y., Wong, H. K., Suzuki, T., Uchikawa, C., Kotliarov, Y., Uchida, K., Nagao, Y., Nagaoka, U., Tamaoka, A., Oyanagi, K., Oyama, F., Nukina, N. (2005) Decreased expression of hypothalamic neuropeptides in Huntington disease transgenic mice with expanded polyglutamine-EGFP fluorescent aggregates. J. Neurochem. 93, 641-653 and Sheng, G., Chang, G. Q., Lin, J. Y., Yu, Z. X., Fang, Z. H., Rong, J., Lipton, S. A., Li, S. H., Tong, G., Leibowitz, S. F., Li, X. J. (2006) Hypothalamic huntingtin-associated protein 1 as a mediator of feeding behavior. Nat. Med. 12, 526-533). Hap1 protein levels were found to be significantly decreased in the hypothalamus (−37%), the nucleus tractus solitarius located in the medulla oblongata (−53%), the midbrain ventral area (−35%) and in the locus coeruleus and the pons (−27%). Htt protein levels were significantly decreased in the hypothalamus (−32%) and the hippocampus (−56%). To confirm these results, the inventors have microdissected brain areas of interest from additional MeCP2-deficient (n=3) and wild type (n=4) mice and extracted total proteins to perform western blotting. Quantification of the western blot results showed low levels of Hap1a (−76%) and Hap1b isoforms (−77%) in the midbrain ventral area of MeCP2-deficient mice. While there was clearly a tendency for the Hap1b isoform to be reduced (−76%), they have failed to find a statistically significant difference in the hypothalamus because of high inter-individual variability. In the cortex, Hap1a isoform is reduced by 51%. One unexpected finding was that the Hap1b isoform was totally absent from the cortex of MeCP2-deficient mice. Reduced Htt protein levels were reported in the cortex (−68%), the ventral midbrain dopaminergic area (−55%) and the pons (−42%). These results indicate that MeCP2 deficiency leads to an overall decrease in Htt and Hap1 protein levels throughout the mouse brain.

### Example 5

### Abnormal Bdnf Protein Distribution Between the Striatum and Cortex in MeCP2-Deficient Mice

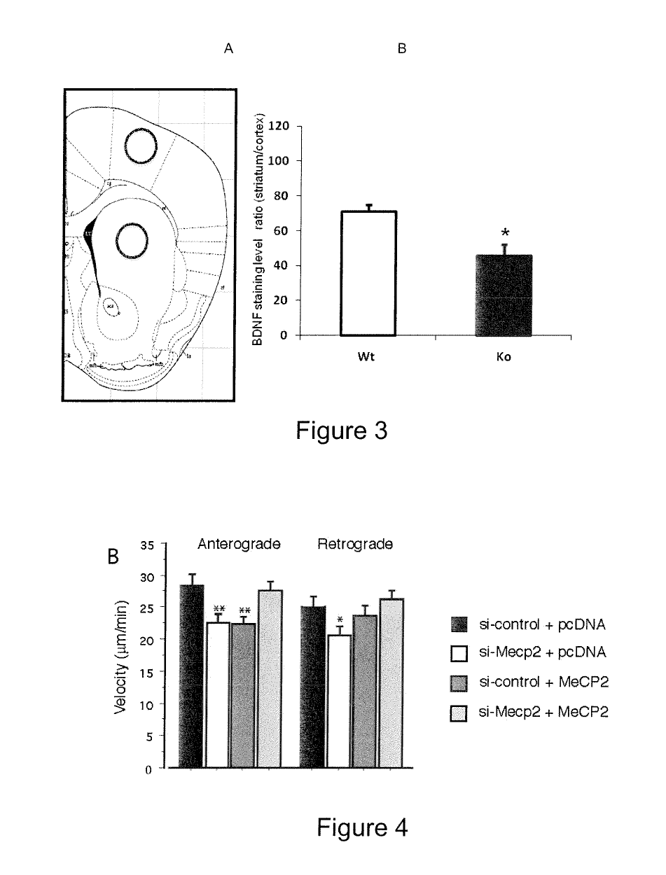

After finding an alteration of Bdnf trafficking in the brain of MeCP2-deficient mice, the inventors have continued investigations in situ to determine whether Bdnf trafficking was altered in the brain of MeCP2-deficient mice. It has been established that Bdnf plays a critical role in the maturation and the metabolism of striatal neurons, even though there is virtually no Bdnf mRNA in striatal cells (Altar, C. A., Cai, N., Bliven, T., Juhasz, M., Conner, J. M., Acheson, A. L., Lindsay, R. M., Wegand, S. J. (1997) Anterograde transport of brain-derived neurotrophic factor and its role in the brain. Nature., 389, 856-60 and Altar, C. A., DiStefano, P. S. (1998) Neurotrophin trafficking by anterograde transport. Trends Neurosci. 21, 433-437). Bdnf protein is translated outside the striatum and is actively transported by an anterograde mechanism into this region of the brain. They have therefore quantified Bdnf staining of the site of translation (the cortex) and the target site (the striatum), and have found that in the absence of MeCP2, Bdnf staining level was reduced in the cortex (−30%) and the striatum (−44%). In wild-type animals, striatal Bdnf accounts for 71% of Bdnf in the cortex. In MeCP2-deficient animals, striatal Bdnf accounts for 45% of cortical Bdnf, suggesting that the transport of Bdnf is altered in vivo when MeCP2 is absent (FIG. 3B).

### Example 6

### MeCP2 Silencing Alters Bdnf Vesicular Velocity

Experiments with rats have been carrying out to determine whether a modification of MeCP2 alters Bdnf trafficking in vivo. The inventors have monitored Bdnf-cherry dynamics in rat cortical neurons under- or overexpressing MeCP2. Rat cortical neurons were electroporated with an inactivating MeCP2 siRNA, a control scrambled RNA, a MeCP2-expressing vector or an empty vector. After three days in culture, MeCP2 levels were assessed by immunofluorescence and the nuclear intensity of the MeCP2 signal was quantified. In parallel, they have analyzed the effect of MeCP2 silencing and overexpression on the dynamics of Bdnf-cherry containing vesicles by fast video-microscopy. The transfection of MeCP2 siRNA caused a reduction in MeCP2 levels, as was expected (−33.1%). More interestingly, the MeCP2 silencing caused a decrease in both anterograde (−20.6%) and retrograde (−17.9%) velocity of Bdnf-cherry containing vesicles (FIG. 4). The transfection of the MeCP2-expressing vector alone caused a decrease only in anterograde velocity (−21.4%) of Bdnf-cherry containing vesicles (FIG. 4). To confirm the specificity of the siRNA approach, the inventors have co-transfected MeCP2 siRNA and the MeCP2-expressing vector in a rescue experiment and have found that on transfection of the plasmid expressing the MeCP2 protein, the velocity of Bdnf-cherry containing vesicles was completely restored to control levels (FIG. 4). To see whether the alteration of Bdnf trafficking may be caused indirectly by a modification of microtubule stabilization, they have stained the microtubules with tubulin and acetylated tubulin and observed their architecture. They were not able to show any effect on microtubule organization when MeCP2 levels were modified. Additionally, they were not able to find that under or overexpression of MeCP2 altered the gross morphology of transfected neurons, as shown by the co-eletroporation with cytoplasmic GFP.

### Example 7

### MeCP2 Silencing Alters App Vesicular Velocity

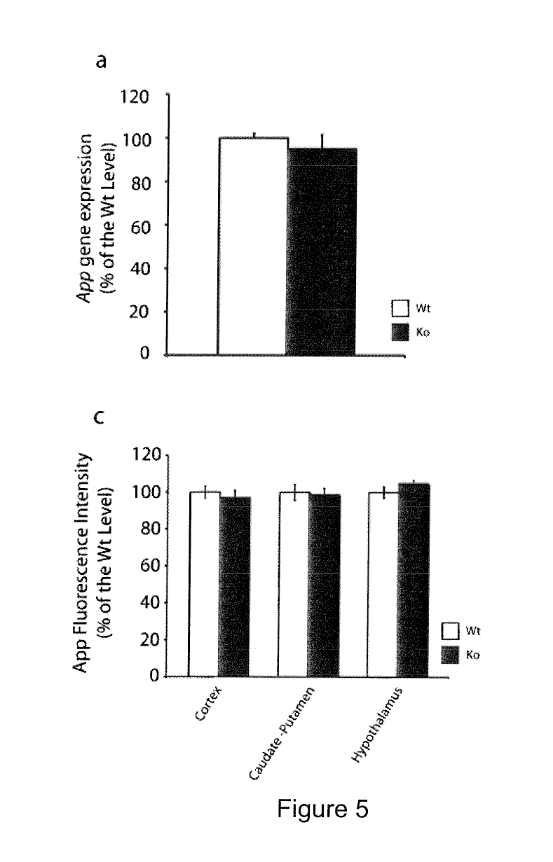

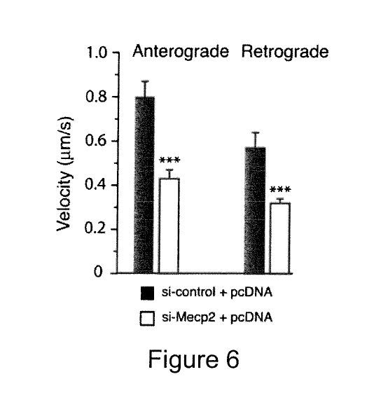

The inventors have repeated the in vivo trafficking experiments of example 6 using amyloid precursor protein (App)-YFP containing vesicles. They have shown that App is not a direct Mecp2 target since they did not found any deregulation at the mRNA and the protein levels (FIG. 5), Moreover, App trafficking is strongly Htt/Hap1-dependent (McGuire et al, 2006 J. Biol. Chem. 281; Colin, E. et al, 2008 EMBO J. 27). Their results clearly showed a deregulation of in vivo App trafficking due to the silencing of Mecp2 (FIG. 6)

### Example 8

### In Vivo Administration of FK506 (Tacrolimus) and/or Cystamine

FK506 and/or cystamine are provided daily and chronically to the in MeCP2-deficient mice. Motor and respiratory evaluations are performed at different developmental stages in order to determine if there is any improvement.
Cysteamine treatment improve lifespan and motor deficits in Mecp2-deficient mice:

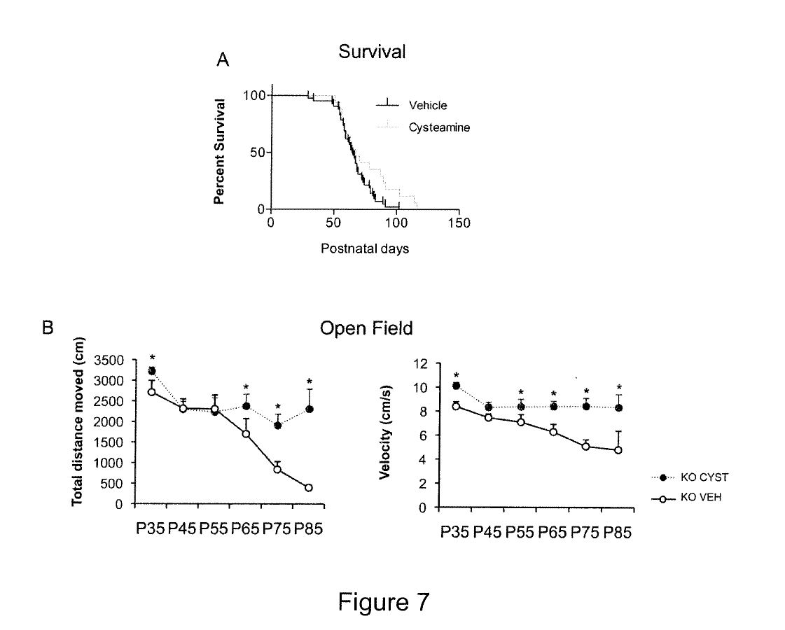

The inventors found that cysteamine oral treatment significantly lengthened the life span of Mecp2-deficient mice (vehicle group: 65±2.2 days; cysteamine group: 74.8±5.2 days; p<0.05, Kaplan-Meir log-rank test) (FIG. 7A). Therefore, the inventors evaluated the impact of this treatment on the locomotion using an open field arena (FIG. 7B-C). Cysteamine treatment significantly improved locomotion in Mecp2-deficient mice as shown by the statistical improvement in both the total distance moved and the velocity (especially in the later stages). Cysteamine treatment was potent to delay the aggravation of motor symptoms compared to the placebo group that exhibited a progressive but marked increase of these symptoms. Consequently, differences between cysteamine treated and the placebo group increased progressively but were particularly evident in later stages both for the total distance (P55: +3%, P65: +40%; P75: +128%, P85: +485%, p<0.05) and the velocity (P55: +18%, P65: +33%; P75: +65%, P85: +74%, p<0.05). These results indicate that a chronic administration of cysteamine is able to improve the performance of Mecp2-deficient mice in paradigms evaluating the voluntary motor behavior.

1. Cystamine or cysteamine, or a salt thereof, for use in treating a MECP2-associated disorder in a patient.

2. Cystamine or cysteamine for use in treating a MECP2-associated disorder according to claim 1, wherein the MECP2-associated disorder is selected from the group consisting of Rett syndrome, autism, Pervasive development disorder, non-syndromic mental retardation, idiopathic neonatal encephalopathy and idiopathic cerebral palsy.

3. Cystamine or cysteamine for use in treating a MECP2-associated disorder according to claim 2, wherein the MECP2-associated disorder is Rett syndrome.

4. Cystamine or cysteamine for use in treating a MECP2-associated disorder according to any of claims 1 to 3, for use in combination with another pharmaceutically active compound.

5. A calcineurin inhibitor for use in treating a MECP2-associated disorder in a human patient.

6. The calcineurin inhibitor for use in treating a MECP2-associated disorder according to claim 5, which is a macrolide.

7. The calcineurin inhibitor for use in treating a MECP2-associated disorder according to claim 5 or 6, which is selected from the group consisting of calcipressins, tacrolimus and tacrolimus analogs, cyclosporine A and cyclosporine A analogs, LxPV proteins, 2,6-diaryl-substitued pyrimidine derivatives and FK506-binding proteins

8. The calcineurin inhibitor for use in treating a MECP2-associated disorder according to claim 7, which is selected from the group consisting of calcipressin 1, calcipressin 2, calcipressin 3, tacrolimus, ascomycin, sirolimus, pimecrolimus, cyclosporine A, voclosporine, LxPVc1, LxPVc2, LxPVc3, 6-(3,4-dichloro-phenyl)-4-(N,N-dimethylaminoethylthio)-2-phenyl-pyrimidine and FK506-binding protein 8.

9. The calcineurin inhibitor for use in treating a MECP2-associated disorder according to claim 8, which is tacrolimus.

10. The calcineurin inhibitor for use in treating a MECP2-associated disorder according to claim 8, which is cyclosporin A.

11. The calcineurin inhibitor for use in treating a MECP2-associated disorder according to any of claims 5 to 9, wherein the MECP2-associated disorder is selected from the group consisting of Rett syndrome, autism, Pervasive development disorder, non-syndromic mental retardation, idiopathic neonatal encephalopathy and idiopathic cerebral palsy.

12. The calcineurin inhibitor for use in treating a MECP2-associated disorder according to claim 11, wherein the MECP2-associated disorder is Rett syndrome.

13. The calcineurin inhibitor for use in treating a MECP2-associated disorder according to any of claims 5 to 12, for use in combination with another pharmaceutically active compound.
